(19) United States
(12) Patent Application Publication
(10) Pub. No.: US 2019/0117822 A1
(43) Pub. Date: Apr. 25, 2019

(54) Adhesive Bandage with Nonprescription Skincare Medication

(71) Applicant: Donald Raven, SR., Florissant, MO (US)

(72) Inventor: Donald Raven, SR., Florissant, MO (US)

(21) Appl. No.: 16/167,511

(22) Filed: Oct. 22, 2018

Related U.S. Application Data
(60) Provisional application No. 62/574,894, filed on Oct. 20, 2017.

(51) Int. Cl. A61L 15/44; A61F 13/00

(52) U.S. Cl. CPC A61L 15/44; A61F 13/00063; A61L 2300/406; A61L 2300/41; A61F 2013/00655; A61L 2300/602; A61L 2300/412; A61F 2013/00421; A61F 2013/00561; A61L 2300/402

(57) ABSTRACT

An adhesive bandage with nonprescription skincare medication includes a flexible strip, a pad retaining layer, a flexible pad, a nonprescription skincare medication, and a pull tab. The nonprescription skincare medication is infused into the flexible pad as the flexible pad is concentrically enclosed in between the flexible strip and the pad retaining layer. An outer edge of the pad retaining layer perimetrically connected a middle section of the flexible strip, wherein an inner edge of the pad retaining layer delineates an opening. The pull tab is hermetically attached to the pad retaining layer and encloses the opening so that the nonprescription skincare medication can be protected from drying out.

The current application claims a priority to the U.S. Provisional Patent application Ser. No. 62/574,894 filed on Oct. 20, 2017. The current application is filed on Oct. 22, 2017 while Oct. 20, 2017 was on a weekend.

### FIELD OF THE INVENTION

The present invention relates generally to adhesive bandage. More specifically, the present invention is an adhesive bandage with nonprescription skincare medication that is infused into a pad of the adhesive bandage.

### BACKGROUND OF THE INVENTION

Hiking and camping are common outdoor recreational activities enjoyed by the general public. There are many benefits associated with hiking and camping, ranging from improved health to increased appreciation and awareness of nature and its bounty. Despite the various benefits attributed to hiking and camping, there do exists some downfalls that are associated with hiking and camping. The most common downfall associated with hiking and camping, which can be the main reason why some people are apprehensive of camping and hiking, is the prevalence of the risk of receiving insect bites. It is almost guaranteed that any individual hiking and/or camping will receive insect bites. However, there are methods and devices that can prevent insect bites from occurring or alleviate insect bites when they occur. A common device to prevent insect bites would be an insect repellent spray that is applied to the user's skin and/or body. However, insect repellent sprays can contain unpleasant smells that are intolerable to the user. Also, some insect repellent sprays contain harmful chemicals that can be hazardous to the user's health or damaging to the user's clothing. Although insect repellent spray is a popular device by many people, other devices exist to address the issues of insect repellent spray, but these devices are too cumbersome to use and are not capable of being mobile enough for the user who is hiking and/or camping. For instance, there exists an insect lamp capable of attracting insects and zapping them when they approach the device. However, this device forces the user to stay confined to an area near the insect lamp, which defeats the purpose of camping and/or hiking: to be mobile in the outdoor environment. There does exists some home remedies that claim to assist users in alleviating insect bites as they occur. A common practice would be to apply some rubbing alcohol to the infected skin where the insect bite occurs. This helps in cooling the infected area so the swelling from an insect bite can reduced. However, this common practice of applying rubbing alcohol is not efficient or practical, since the user must constantly reapply a fresh application of the rubbing alcohol when the previous application has dried out. Also, the user could have a difficulty in performing this practice of applying rubbing alcohol on their own.

An objective of the present invention is to provide users with an adhesive bandage that contains rubbing alcohol or other similar types of nonprescription skincare medications. The present invention intends to resolve issues involved with insect repellent spray and other similar devices as mentioned previously. The present invention is less harmful than aforementioned solutions because the present invention does not contain any harmful chemicals that can be damaging to the user's health and/or clothing. Also, the present invention reduces the amount of unpleasant smells to the user by making the invention only applicable to parts of the user's body where the insect bite occurs. The present invention allows the user to still be active and mobile since the present invention does restrict the user's movement and/or mobility, and/or the present invention does confine the user to specific zone or area while addressing the insect bite. Finally, the present invention is easier to utilize than the common practice of applying rubbing alcohol or nonprescription skincare medications since the present invention combines the rubbing alcohol or nonprescription skincare medications, with an adhesive bandage without drying out the rubbing alcohol or nonprescription skincare medications.

### BRIEF DESCRIPTION OF THE DRAWINGS

FIG. 1 is a front perspective view of the present invention, showing the front surface of the flexible strip.

FIG. 2 is a rear perspective view of the present invention, showing the pull tab and sections of the flexible strip.

FIG. 3 is a rear view of the flexible strip of the present invention, showing three sections of the flexible strip and showing the plane upon which a cross sectional view is taken shown in FIG. 4.

FIG. 4 is a cross section view of the present invention taken along line A-A of FIG. 3, showing the detail view is taken shown in FIG. 5.

FIG. 5 is a detail view of the present invention taken within section B of the FIG. 4.

FIG. 6 is a rear perspective view of the present invention, showing the pull tab, the first adhesive layer, and the second adhesive layer as the first removable film and second removable film are removed.

FIG. 7 is a rear perspective view of the present invention, showing the flexible pad, the first adhesive layer, and the second adhesive layer as pull tab, the first removable film, and the second removable film are removed.

FIG. 8 is a shamanic view showing the different types of medications of the quantity of nonprescription skincare medication of the present invention.

### DETAIL DESCRIPTIONS OF THE INVENTION

All illustrations of the drawings are for the purpose of describing selected versions of the present invention and are not intended to limit the scope of the present invention.

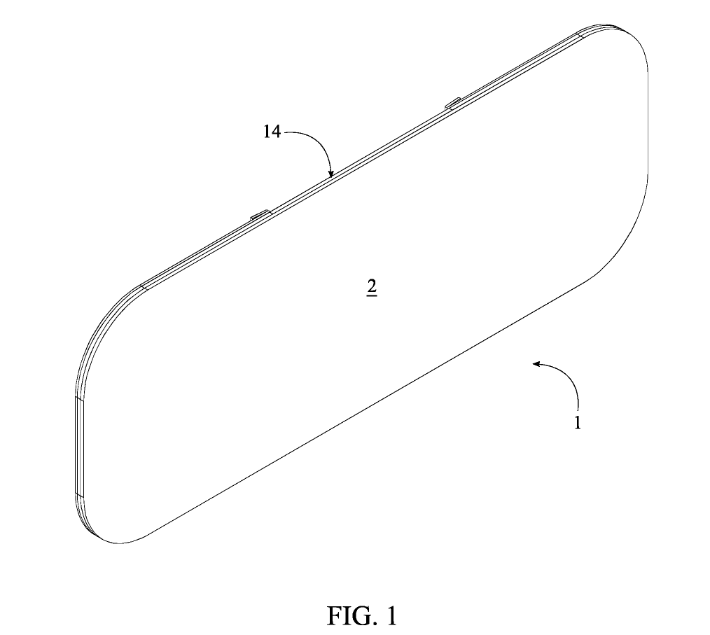

The present invention is an adhesive bandage that contains a dosage of over-the-counter skincare medication so that the users can utilize the present invention for insect bites, wound cleaning, skin moistening, and other types of skin disorders that require medication. In other words, the present invention is a combination of an adhesive bandage and over-the-counter medication that are combined into an individual and sealed package. The present invention comprises a flexible strip 1, a pad retaining layer 14, a flexible pad 17, a quantity of nonprescription skincare medication 19, and a pull tab 20 as shown in FIG. 1-2 and FIG. 7.

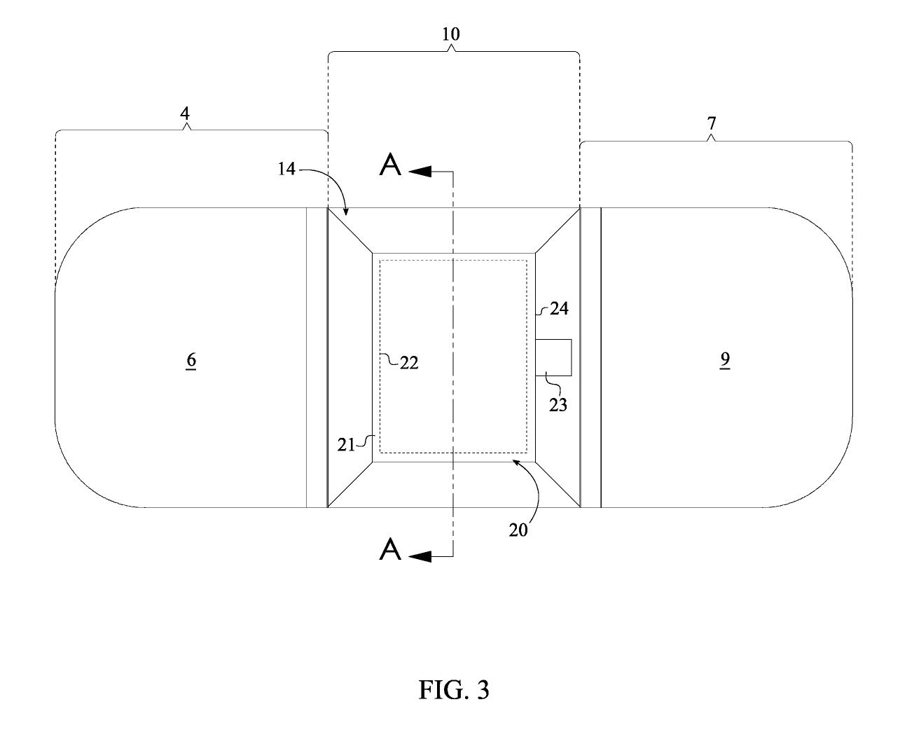

The flexible strip 1 functions as the base member for the present invention as the rest of the components of the present invention are configured onto the flexible strip 1. In reference to FIG. 1-4, the flexible strip 1 comprises a front surface 2, a rear surface 3, a first distal section 4, a second distal section 7, and a middle section 10. The first distal section 4 that allows the present invention to be adhered from one side is terminally connected to the middle section 10. The second distal section 7, that allows the present invention to be adhered from the opposite side, is terminally connected to the middle section 10 and positioned opposite of the first distal section 4. The front surface 2 extends along the first distal section 4, the middle section 10, and the second distal section 7 delineating a flat surface. Similarly, the rear surface 3 extends along the first distal section 4, the middle section 10, and the second distal section 7 delineating a flat surface that is positioned opposite of the front surface 2. Furthermore, the front surface 2 orients away from the user's body and the rear surface 3 orients toward the user's body when the present invention is utilized.

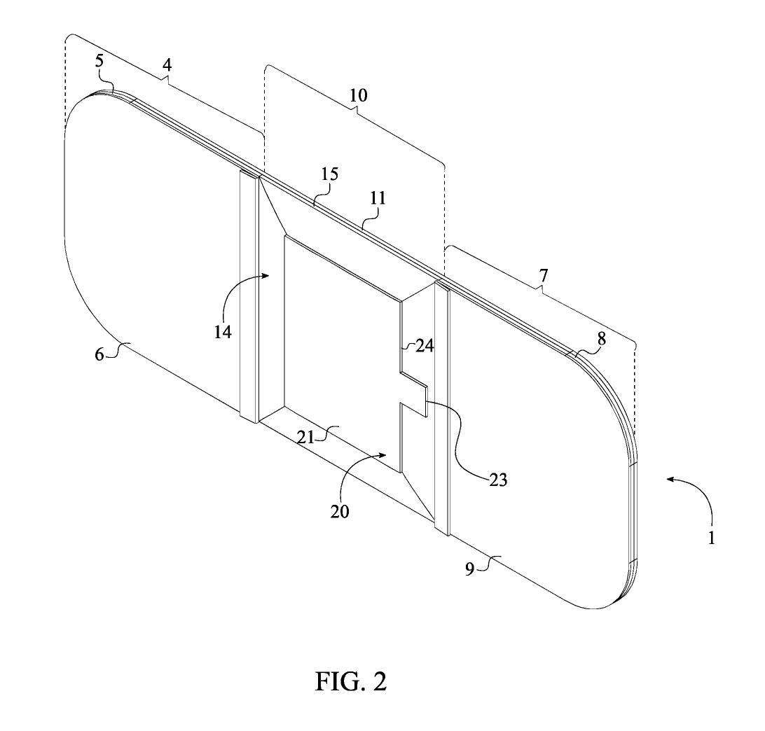

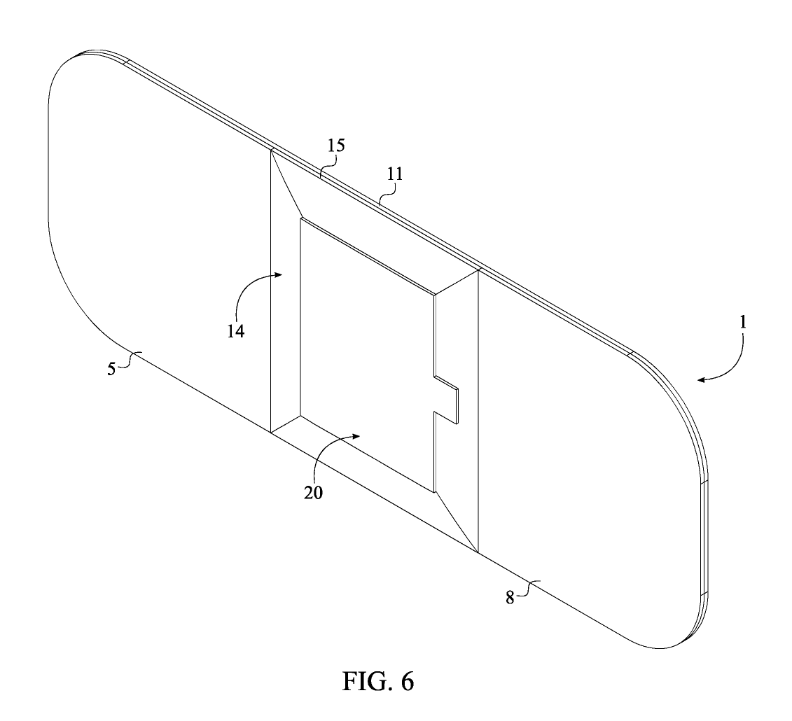

In reference to FIG. 2 and FIG. 6, the present invention further comprises a first adhesive layer 5 and a first removable film 6. The first adhesive layer 5 provides adhesive properties for the first distal section 4 so that the first distal section 4 can be adhered to the user's skin. More specifically, the first adhesive layer 5 is coextensively positioned with the first distal section 4 and superimposed onto the rear surface 3 of the first distal section 4. The first removable film 6 is attached onto the first distal section 4 by the first adhesive layer 5 so that the first removable film 6 can protect the adhesive properties of the first adhesive layer 5 until the present invention is ready to be utilized.

In reference to FIG. 2 and FIG. 6, the present invention further comprises a second adhesive layer 8 and a second removable film 9. The second adhesive layer 8 provides adhesive properties for the second distal section 7 so that the second distal section 7 can be adhered to the user's skin. More specifically, the second adhesive layer 8 is coextensively positioned with the second distal section 7 and superimposed onto the rear surface 3 of the second distal section 7. The second removable film 9 is attached onto the second distal section 7 by the second adhesive layer 8 so that the second removable film 9 can protect the adhesive properties of the second adhesive layer 8 until the present invention is ready to be utilized.

The pad retaining layer 14 forms a pouch within the present invention as the flexible pad 17 is positioned in between the pad retaining layer 14 and the flexible strip 1 thus sandwiching the flexible pad 17 within. As a result, the flexible pad 17 hermetically positioned within the pouch until the flexible pad 17 is accessed through the pull tab 20.

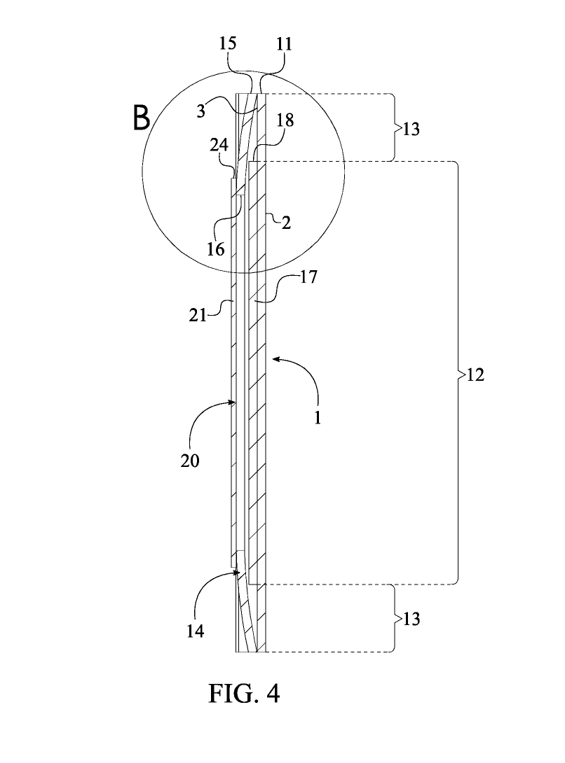

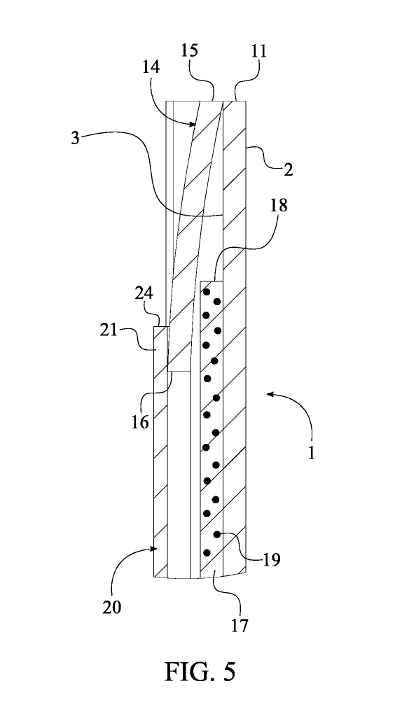

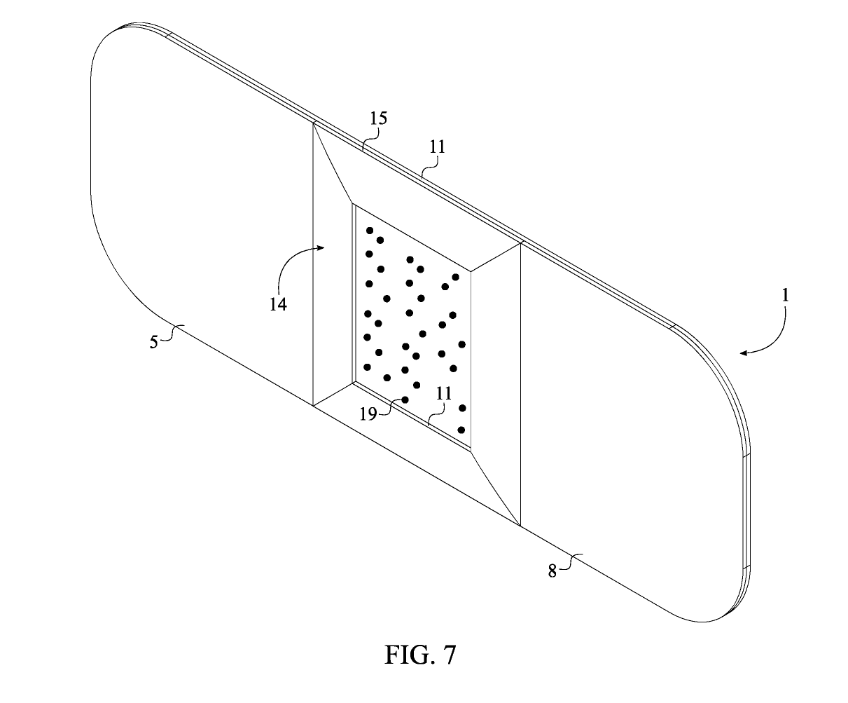

In reference to FIG. 7, the flexible pad 17 that is infused with the quantity of nonprescription skincare medication 19 to treat many different types of skin deceases. The flexible pad 17 is concentrically connected to the rear surface 3 of the flexible strip 1 so that the rear surface 3 can be positioned adjacent to the user's skin. In reference to FIG. 4-5, an outer edge 15 of the pad retaining layer 14 is perimetrically connected to an outer edge 11 of the middle section 10 so that the pouch can be formed within the present invention. Furthermore, an inner edge 16 of the pad retaining layer 14 delineates an opening for the pouch, wherein the opening is hermetically covered through the pull tab 20.

The flexible strip 1 can be made of a plastic and/or fabric material that is flexible, stretchable, durable, and sterile. The flexibility of the flexible strip 1 and the flexible pad 17 allow the present invention to be contoured along the user's body when utilized. The front surface 2 and rear surface 3 of the middle section 10 are impermeable surfaces. More specifically, the front surface 2 and the rear surface 3 being impermeable are important to preserve the quantity of nonprescription skincare medication 19 within the present invention, so that the flexible pad 17 can retain the medicinal qualities of the quantity of nonprescription skincare medication 19. Furthermore, the front surface 2 and the rear surface 3 for the first distal section 4 are permeable surfaces so that the first distal section 4 can ventilate the user's skin when the present invention is adhered. Similarly, the front surface 2 and the rear surface 3 for the second distal section 7 are permeable surfaces so that the second distal section 7 can ventilate the user's skin when the present invention is adhered

In reference to FIG. 4, the middle section 10 further comprises an inner section 12 and an outer section 13. More specifically, the inner section 12 and the outer section 13 are concentrically positioned with each other. These two different sections of the middle section 10 illustrate different surface area so that positioning of the flexible pad 17 can be clearly explained in relation to the inner section 12 and the outer section 13. The outer section 13 is peripherally positioned around the inner section 12 so that the flexible pad 17 can be coextensively positioned with the inner section 12. In other words, an outer edge 18 of the flexible pad 17 is perimetrically positioned within the outer edge 15 of the pad retaining layer 14 and the inner edge 16 of the pad retaining layer 14 thus preventing the flexible pad 17 from dislodging within the present invention. As a result, an outermost parameter of the flexible pad 17 positions behind the pad retaining layer 14 while an innermost surface area of the flexible pad 17 is accessible for treatments.

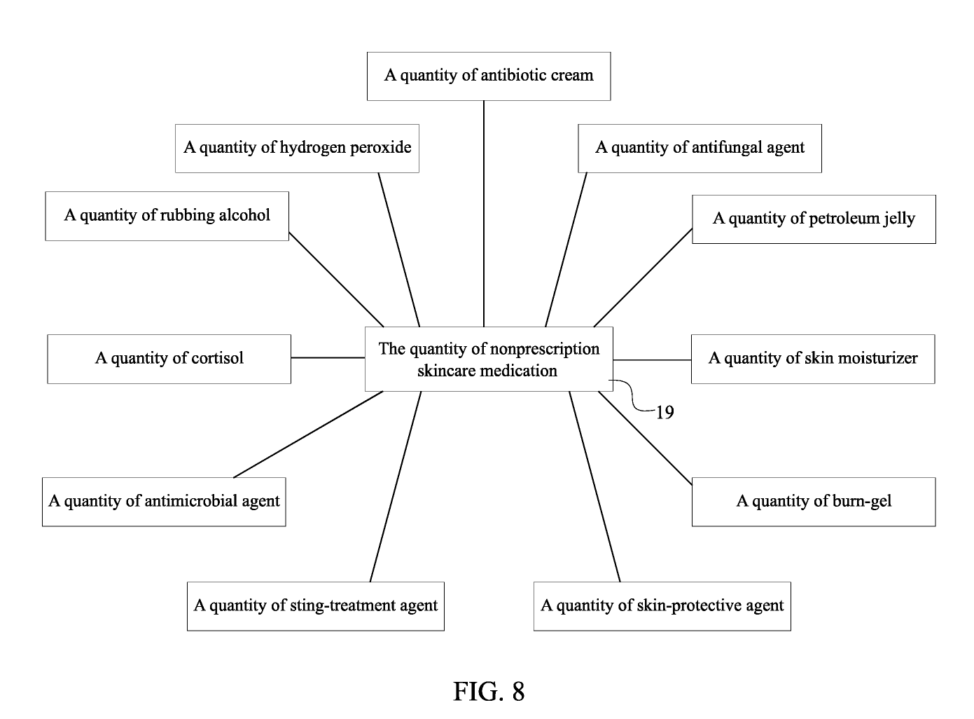

In reference to FIG. 8, the quantity of nonprescription skincare medication 19 can be a quantity of rubbing alcohol so that the present invention can be utilized as sanitizer and disinfecting pads. The quantity of rubbing alcohol generally contains 0-70% solution of isopropyl alcohol in water as water functions as the drying aid.

In reference to FIG. 8, the quantity of nonprescription skincare medication 19 can be a quantity of hydrogen peroxide so that the present invention can be utilized as disinfecting agent for wounds, insect bites, any other types of skin problems that require antiseptics.

In reference to FIG. 8, the quantity of nonprescription skincare medication 19 can be a quantity of cortisol so that the present invention can be utilized as anti-itch medication. More specifically, the quantity of cortisol, also known as hydrocortisone, is a non-greasy cream. The quantity of cortisol within the present invention can penetrate quickly and relive skin irritation, rashes, inflammation, redness, and insect bites.

In reference to FIG. 8, the quantity of nonprescription skincare medication 19 can be a quantity of antibiotic cream so that the present invention can be utilized as antiseptics. More specifically, the quantity of antibiotic cream generally contains neomycin, polymyxin B, and bacitracin so that the quantity of antibiotic cream within the present invention can be utilized to prevent skin infections.

In reference to FIG. 8, the quantity of nonprescription skincare medication 19 can be a quantity of skin moisturizer. More specifically, the quantity of skin moisturizer within the present invention can be utilized for protecting, moisturizing, and lubricating skin so that skin can be protected from water loss, pathogens, dirt, and damage.

In reference to FIG. 8, the quantity of nonprescription skincare medication 19 can be a quantity of antimicrobial agent so that the present invention can be utilized to kill microorganism or stops their growth. More specifically, the quantity of antimicrobial agent within the present invention can be utilized to treat tinea pedis, also known as athlete's foot, that is a skin infection of the feet caused by fugus.

In reference to FIG. 8, the quantity of nonprescription skincare medication 19 can be a quantity of burn-gel so that the present invention can be utilized against minor skin burns. More specifically, the quantity of burn-gel within the present invention can be utilized to treat sun burn, itching, shingles, hives, chaffing, nerve pains, minor cuts, scrapes, and insect bites.

In reference to FIG. 8, the quantity of nonprescription skincare medication 19 can be a quantity of petroleum jelly so that the present invention can be utilized as topical ointment. More specifically, the quantity of petroleum jelly within the present invention can be utilized as cosmetic purposes and treatments for toenail fungus, non-sexually transmitted genital rashes, nosebleeds, diaper rash, and chest cold.

In reference to FIG. 8, the quantity of nonprescription skincare medication 19 can be a quantity of antifungal agent so that the present invention can be utilized eliminates fungal pathogens from a host with minimal toxicity to the host. More specifically, the quantity of antifungal agent within the present invention can be utilized to skin diseases such as athlete's foot, ringworm, candidiasis, and serious systemic infections.

In reference to FIG. 8, the quantity of nonprescription skincare medication 19 can be a quantity of skin-protective agent so that the present invention can be utilized as dealing agent for skin. More specifically, the quantity of skin-protective agent within the present invention can be utilized to treat diaper rash, dry or chafed skin, and minor cuts or burns as the quantity of skin-protective agent moisturizes and seals the skin.

In reference to FIG. 8, the quantity of nonprescription skincare medication 19 can be a quantity of sting-treatment agent so that the present invention can be utilized against insect bites or stings. More specifically, the quantity of sting-treatment agent within the present invention can be utilized against bee, wasp, and mosquito sting as the quantity of sting-treatment agent alleviates the itchiness and pain of bites and stings.

In reference to FIG. 8, the pull tab 20 that seals the flexible pad 17 comprises a body 21, an adhesive strip 22, and at least one tab 23. More specifically, adhesive strip 22 is perimetrically superimposed within an outer edge 24 of the body 21 so that the adhesive strip 22 can attached the body 21 to pad retaining layer 14. The at least one tab 23 is terminally connected to the outer edge 24 of the body 21 as the outer edge 24 of the body 21 is perimetrically positioned around the inner edge 16 of the pad retaining layer 14. Resultantly, the body 21 is hermetically attached onto the pad retaining layer 14 by the adhesive strip 22 so that the flexible pad 17 can be hermetically sealed within the flexible strip 1 and the pad retaining layer 14. In order to easily peel off the pull tab 20, the at least one tab 23 is oriented away from the outer edge 24 of the body 21 thus allowing the user to easily grasp only the at least one tab 23.

Due the airtight connection between the flexible strip 1 and the body 21 of the pull tab 20 through the adhesive strip 22, the present invention is able to eliminate the traditional packaging that envelops the traditional adhesive bandages thus reducing manufacturing cost. Furthermore, the different medication types of the quantity of nonprescription skincare medication 19 provide a variety of medicinal options for the user of the present invention. When the user is ready to use the present invention, the pull tab 20 is detached from the pad retaining layer 14 thus exposing the quantity of nonprescription skincare medication 19. The first removable film 6 and the second removable film 9 are then respectively removed from the first distal section 4 and the second distal section 7 thus exposing the first adhesive layer 5 and the second adhesive layer 8. The rear surface 3 is then placed adjacent to the user's skin so that the flexible pad 17 can be placed upon the infected area while the first adhesive layer 5 and the second adhesive layer 8 secured the present invention to the user's skin.

Although the invention has been explained in relation to its preferred embodiment, it is to be understood that many other possible modifications and variations can be made without departing from the spirit and scope of the invention as hereinafter claimed.

What is claimed is:

1. An adhesive bandage with nonprescription skincare medication comprises:
a flexible strip;
a pad retaining layer;
a flexible pad;
a quantity of nonprescription skincare medication;
a pull tab;
the flexible strip comprises a first distal section, a second distal section, and a middle section;
the first distal section being terminally connected to the middle section;
the second distal section being terminally connected to the middle section, opposite of the first distal section;
an outer edge of the pad retaining layer being perimetrically connected to an outer edge of the middle section;
the quantity of nonprescription skincare medication being infused into the flexible pad;
the flexible pad being concentrically connected a rear surface of the flexible strip;
the flexible pad being positioned in between the flexible strip and the pad retaining layer;
the pull tab being perimetrically positioned around an inner edge of the pad retaining layer; and
the pull tab being hermetically attached onto the pad retaining layer, opposite of the flexible strip.

2. The adhesive bandage with nonprescription skincare medication as claimed in claim 1 comprises:
the flexible strip further comprises a front surface;
the front surface being positioned opposite of the rear surface;
the front surface and the rear surface for the middle section being impermeable surfaces;
the front surface and the rear surface for the first distal section being permeable surfaces; and
the front surface and the rear surface for the second distal section being permeable surfaces.

3. The adhesive bandage with nonprescription skincare medication as claimed in claim 1 comprises:
the middle section further comprises an inner section and an outer section;
the inner section and the outer section being concentrically positioned with each other;
the outer section being peripherally positioned around the inner section;
the flexible pad being coextensively positioned with the inner section; and
an outer edge of the flexible pad being perimetrically positioned within the outer edge of the pad retaining layer and the inner edge of the pad retaining layer.

4. The adhesive bandage with nonprescription skincare medication as claimed in claim 1 comprises:
a first adhesive layer;
a first removable film;
the first adhesive layer being coextensively positioned with the first distal section;
the first adhesive layer being superimposed onto the rear surface of the first distal section; and
the first removable film being attached onto the first distal section by the first adhesive layer.

5. The adhesive bandage with nonprescription skincare medication as claimed in claim 1 comprises:
a second adhesive layer;
a second removable film;
the second adhesive layer being coextensively positioned with the second distal section;
the second adhesive layer being superimposed onto the rear surface of the second distal section; and
the second removable film being attached onto the second distal section by the second adhesive layer.

6. The adhesive bandage with nonprescription skincare medication as claimed in claim 1 comprises:
the pull tab comprises a body, an adhesive strip, and at least one tab;
the adhesive strip being perimetrically superimposed within an outer edge of the body;
the at least one tab is terminally connected to the outer edge of the body;
the at least one tab being oriented away from the outer edge of the body.
the outer edge of the body is perimetrically positioned around the inner edge of the pad retaining layer; and
the body is hermetically attached onto the pad retaining layer by the adhesive strip.

7. The adhesive bandage with nonprescription skincare medication as claimed in claim 1, wherein the quantity of nonprescription skincare medication is a quantity of rubbing alcohol.

8. The adhesive bandage with nonprescription skincare medication as claimed in claim 1, wherein the quantity of nonprescription skincare medication is a quantity of hydrogen peroxide.

9. The adhesive bandage with nonprescription skincare medication as claimed in claim 1, wherein the quantity of nonprescription skincare medication is a quantity of cortisol.

10. The adhesive bandage with nonprescription skincare medication as claimed in claim 1, wherein the quantity of nonprescription skincare medication is a quantity of antibiotic cream.

11. The adhesive bandage with nonprescription skincare medication as claimed in claim 1, wherein the quantity of nonprescription skincare medication is a quantity of skin moisturizer.

12. The adhesive bandage with nonprescription skincare medication as claimed in claim 1, wherein the quantity of nonprescription skincare medication is a quantity of antimicrobial agent.

13. The adhesive bandage with nonprescription skincare medication as claimed in claim 1, wherein the quantity of nonprescription skincare medication is a quantity of burn-gel.

14. The adhesive bandage with nonprescription skincare medication as claimed in claim 1, wherein the quantity of nonprescription skincare medication is a quantity of petroleum jelly.

15. The adhesive bandage with nonprescription skincare medication as claimed in claim 1, wherein the quantity of nonprescription skincare medication is a quantity of antifungal agent.

16. The adhesive bandage with nonprescription skincare medication as claimed in claim 1, wherein the quantity of nonprescription skincare medication is a quantity of skin-protective agent.

17. The adhesive bandage with nonprescription skincare medication as claimed in claim 1, wherein the quantity of nonprescription skincare medication is a quantity of sting-treatment agent.
